(19) United States
(12) Patent Application Publication
(10) Pub. No.: US 2022/0240895 A1
(43) Pub. Date: Aug. 4, 2022

(54) PULMONARY ARTERY CATHETER WITH ECHOCARDIOGRAPHY PROBE

(71) Applicant: Northwestern University, Evanston, IL (US)

(72) Inventor: Christopher K. Mehta, Chicago, IL (US)

(21) Appl. No.: 17/589,137

(22) Filed: Jan. 31, 2022

Related U.S. Application Data
(60) Provisional application No. 63/144,006, filed on Feb. 1, 2021.

(51) Int. Cl. A61B 8/00; A61B 8/12; A61B 8/04

(52) U.S. Cl. CPC A61B 8/445; A61B 8/12; A61M 25/10182; A61B 8/4416; A61B 8/04

(57) ABSTRACT

Provided herein are pulmonary artery catheters comprising an echocardiogram probe. Devices herein are capable of taking both hemodynamic and echocardiographic measurements.

### CROSS-REFERENCE TO RELATED APPLICATIONS

The present application claims the priority benefit of U.S. Provisional Patent Application 63/144,006, filed Feb. 1, 2021, which is incorporated by reference in its entirety.

### FIELD

Provided herein are pulmonary artery catheters comprising an echocardiogram probe. Devices herein are capable of taking both hemodynamic and echocardiographic measurements.

### BACKGROUND

A pulmonary artery catheter (PAC) is a thermo dilution catheter that is inserted via a large vein and floated into the pulmonary artery. It is used to obtain hemodynamic measurements, which together with clinical observations indicate how efficiently the heart is functioning. Currently available invasive hemodynamic monitoring devices provide clinical information based on pressure and waveform readings which must be interpreted in the appropriate context by clinicians. However, this physiologic data is limited, particularly in post-surgical patients with complex cardiovascular disease and mechanical circulatory support devices.

Echocardiography is a test that uses sound waves to produce live images of the heart. The image produced is called an echocardiogram. Echocardiography allows for monitoring the function of the heart and its valves. Echocardiography provides visual demonstration of how the left and right atria and ventricles contract, overall heart ejection function, valvular function or dysfunction (i.e. degree of regurgitation or stenosis), appropriate placement and location of intracardiac circulatory support devices, and quality of post-surgical repair. Currently available echocardiograms are difficult to obtain. Transthoracic echocardiogram (TTE) (ultrasound probe on surface of chest) gives limited views, especially in post-surgical patients, because of surgical dressings, body habitus/tissue, and residual blood clot surrounding the heart which may be normal after surgery. In addition, the logistics of obtaining this study can be difficult. Most hospitals do not have technicians available 24 hours, 7 days a week to perform this study. For example, in the middle of the night an echocardiography technician usually needs to be called in from home which delays care. And this only provides a one-time snapshot of the heart. If an intervention is needed (e.g., administering the patient a 500 ml normal saline bolus over one hour), one would again need to call the technician back to assess how the patient performed. This is impractical and delays care in emergent situations. A transesophageal echocardiogram (TEE) is invasive, requiring placement by an experienced provider down the esophagus and therefore only can be used in intubated patients. This also requires specialized expertise and specialized personnel (usually a cardiologist or anesthesiologist) in order to perform. It comes with a risk of esophageal perforation if not performed correctly. In the ICU setting this is considered a very invasive assessment of hemodynamics and is therefore typically not performed. Like TTE, TEE only provides a one-time snapshot of the heart. An intracardiac echocardiogram (ICE) provides the best resolution and visualization of the heart since it is closest to heart chambers; however, it is only indicated for procedural use by interventional cardiologists and electrophysiologists in a monitored cardiac catheterization laboratory, and is only transiently used for particular interventions (i.e., not left in place for continual monitoring).

### SUMMARY

Provided herein are pulmonary artery catheters comprising an echocardiogram probe. Devices herein are capable of taking both hemodynamic and echocardiographic measurements.

In some embodiments, devices herein comprise an ultrasound probe affixed to a catheter, which can be placed in the right ventricle. Such devices can be used for real-time assessment of the right and left ventricular function, ejection fraction, valvular function, and positioning of intracardiac devices. In some embodiments, the devices herein also comprise components for making hemodynamic measurements, and therefore assessments made by ultrasound can be correlated the pressure hemodynamics from the catheter to make a more accurate assessment of the patient's clinical picture. In some embodiments, practitioners use the devices herein to assess the effect of interventions on heart function and base further interventions on visual identification of the heart. In some embodiments, devices herein do not require the specialized personnel required for ICE and TEE and therefore overcome the delays associated with other means of obtaining cardiac ultrasound information.

In some embodiments, provided herein are devices comprising a catheter body having a distal end for placement within a subject and a proximal end configured to reside outside of the subject, the device comprising: (a) a positioning element located adjacent to the distal end of the catheter body and configured for placement of the distal end of the catheter at a desired location within the subject; (b) a distal opening located at the distal end of the catheter body, a distal port extending from the proximal end of the catheter body, and a distal lumen proving fluid communication from the distal opening to the distal port; and (c) an ultrasound transducer (electrocardiogram probe) located 10-20 cm from the distal end of the catheter body, and a monitoring wire connected to the ultrasound transducer extending from the proximal end of the catheter body.

In some embodiments, the distally-located positioning element is an inflatable balloon. In some embodiments, the distally-located positioning element is a sail. In some embodiments, the balloon is connected to an inflation/deflation lumen that extends from the balloon through the length of the catheter (e.g., through a lumen within the catheter) to the proximal end of the catheter. In some embodiments, a connection is provided at the proximal end of the inflation/deflation lumen for attachment to a balloon controller configured for inflating and deflating the balloon. In some embodiments, the proximal end of the inflation/deflation lumen comprises a balloon controller for inflating and deflating the balloon. In some embodiments, a balloon controller is a syringe. In some embodiments, the balloon has an inflation volume of between 0.25 and 2.5 ml (e.g., 0.25 ml, 0.5 ml, 0.75 ml, 1.0 ml, 1.25 ml, 1.5 ml, 1.75 ml, 2.0 ml, 2.25 ml, 2.5 ml, or ranges therebetween (e.g., 0.5-1.5 ml)).

In some embodiments, the distal opening is 0.5-2.0 mm (e.g., 0.5, 0.75, 1.0, 1.25, 1.5, 1.75, 2.0, or ranges therebetween) in diameter. In some embodiments, the distal opening is 1 mm in diameter. In some embodiments, the distal lumen is 0.5-2.0 mm (e.g., 0.5, 0.75, 1.0, 1.25, 1.5, 1.75, 2.0, or ranges therebetween) in diameter. In some embodiments, the distal lumen is 1 mm in diameter. In some embodiments, the distal port is configured for connection to a hemodynamic monitoring system. In some embodiments, the distal port and the distal opening are connected by a lumen within the catheter body.

In some embodiments, the ultrasound transducer located is 14-16 cm (e.g., 14 cm, 14.25 cm, 14.5 cm, 14.75 cm, 15.0 cm, 15.25 cm, 15.5 cm, 15.75 cm, 16.0 cm, or ranges therebetween) from the distal end of the catheter body. In some embodiments, the ultrasound transducer is located 15 cm from the distal end of the catheter body. In some embodiments, the ultrasound transducer is configured for emitting and receive sound waves. In some embodiments, the ultrasound transducer is configured to convert received sound waves into electrical pulses. In some embodiments, the ultrasound transducer is configured to send electrical pulses via the monitoring wire. In some embodiments, the monitoring wire extends from the ultrasound transducer to the proximal end of the device (e.g., through a lumen within the catheter body). In some embodiments, the monitoring wire is configured for connection to a computer system or electrocardiogram monitor. In some embodiments, the monitoring wire places the computer system or electrocardiogram monitor in electric communication with the ultrasound transducer. In some embodiments, the monitoring wire is a coaxial cable.

In some embodiments, devices further comprise a thermistor comprising a temperature-sensitive wire within the catheter body (e.g., within a lumen within the catheter body) that extends from the proximal end of the catheter to a thermistor bead located 2-6 cm (e.g., 2 cm, 2.5 cm, 3 cm, 3.5 cm, 4 cm, 4.5 cm, 5 cm, 5.5 cm, 6 cm, or ranges therebetween) from the distal end of the catheter body. In some embodiments, the proximal end of the thermistor is configured for attachment to a cardiac output monitor.

In some embodiments, devices herein further comprise a proximal opening located 25-35 cm (e.g., 25 cm, 26 cm, 27 cm, 28 cm, 29 cm, 30 cm, 31 cm, 32 cm, 33 cm, 34 cm, 35 cm, or ranges therebetween (e.g., 28-32 cm)) from the distal end of the catheter body, a proximal port extending from the proximal end of the catheter body, and a proximal lumen proving fluid communication from the proximal opening to the proximal port.

In some embodiments, provided herein are methods of cardiac monitoring comprising: (a) placing a catheter device described herein within the heart of a subject such that the distal tip of the catheter body is within the pulmonary artery of the subject and the ultrasound transducer is within the right ventricle of the subject; (b) monitoring hemodynamic pressure using the distal opening, distal lumen, and distal port; and (c) obtaining an echocardiogram using the ultrasound transducer and monitoring wire.

In some embodiments, placing the device comprises: (i) inserting the device into a vein of the subject; (ii) guiding the distal tip of the device through the superior vena cava, right atrium, right ventricle, and into the pulmonary artery of the subject. In some embodiments, the device is inserted into the jugular vein of the subject (or subclavian vein, or femoral vein). In some embodiments, the device is inserted using the Seldinger technique, modified Seldinger technique, or accelerated Seldinger technique (Stoker R, Accelerated Seldinger Technique. Managing Infection Control Magazine, p. 32-36, March 2009; incorporated by reference in its entirety). In some embodiments, placing the device comprises: (iii) expanding the positioning element within the pulmonary artery and allowing the positioning element to occlude a small pulmonary blood vessel. In some embodiments, the positioning element is a balloon and expanding the positioning element comprises inflating the balloon. In some embodiments, the device remains placed in the heart for at least 1 day (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or more, or ranges therebetween (e.g., 8-16 days, 14 days or more, etc.).

In some embodiments, monitoring hemodynamic pressure using the distal opening, distal lumen, and distal port comprises monitoring the pulmonary artery wedge pressure (PAWP).

In some embodiments, methods further comprise monitoring right atrial pressures using the proximal opening, proximal lumen, and proximal port.

In some embodiments, methods further comprise introducing fluids, therapeutics, and/or injectate for cardiac output studies into the right atrium via the proximal opening, proximal lumen, and proximal port.

In some embodiments, methods further comprise monitoring temperature within a chamber of the heart (e.g., right aorta, right ventricle, etc.) via the thermistor.

In some embodiments, provided herein are systems comprising a catheter device described herein and one or more of: (i) a catheter insertion kit; (ii) a hemodynamic monitor subsystem; (iii) an electrocardiogram subsystem; and (iv) a cardiac output monitor. In some embodiments, a catheter insertion kit comprises one or more of a needle, guidewire, sheath introducer, and scalpel. In some embodiments, a hemodynamic monitor subsystem comprises one or more of a oscilloscope or computer-implemented oscilloscope, pressure transducer, fluid-filled pressure bag, rigid pressure tubing, and stopcock or valve. In some embodiments, an electrocardiogram subsystem comprises a computer system or electrocardiogram monitor.

### BRIEF DESCRIPTION OF THE DRAWINGS

FIG. 1. Schematic of an exemplary device herein placed in the heart of a subject such that the distal end of the device resides in the pulmonary artery and the echocardiogram probe resides in the right ventricle.

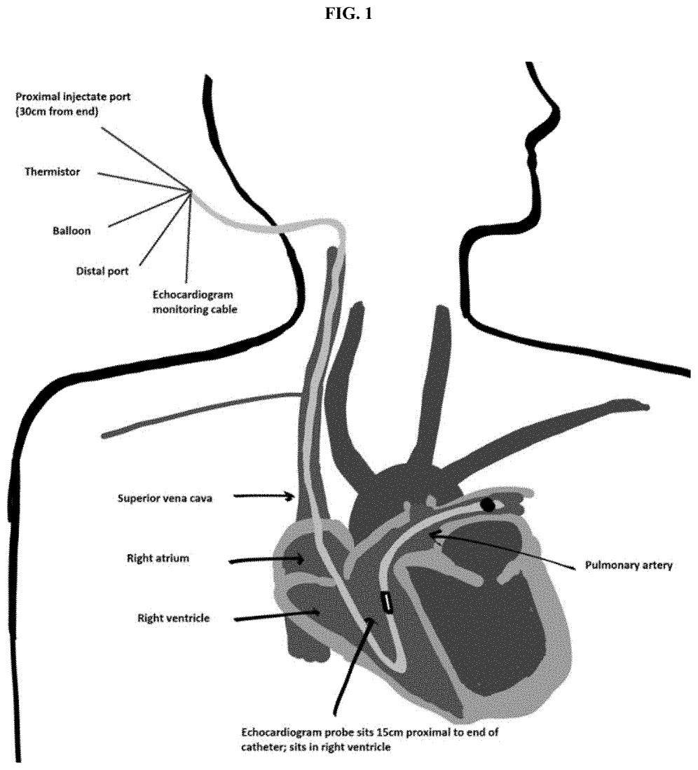

FIG. 2. Schematic of an exemplary device 1 comprising a catheter body 10, a distal opening 21, positioning element 31, ultrasound transducer 41, proximal opening 51, and thermistor 41. In the embodiment depicted, the distal opening 21 is connected to a distal port 22 by a distal lumen 23, the positioning element 31 is connected to controller 32 by a positioning lumen 33, the ultrasound transducer 41 is connected to an ultrasound subsystem 42 (e.g., computer) by a wire 43, the proximal opening 51 is connected to a proximal port 52 by a proximal lumen 53, and the thermistor 61 is connected to a thermistor port 62 by a thermistor wire 63.

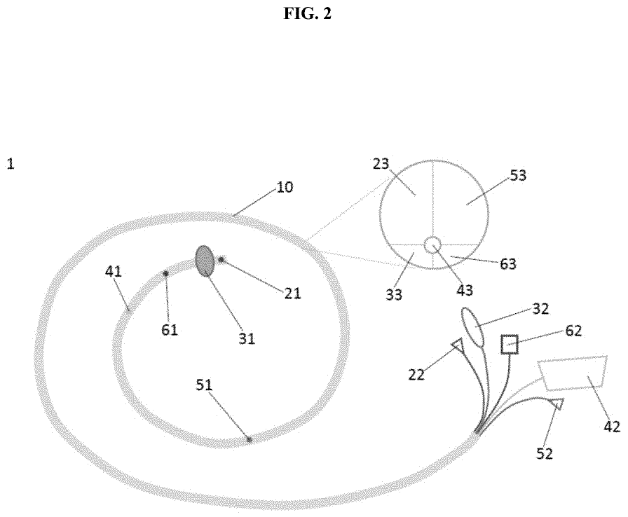

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a biomarker” is a reference to one or more biomarkers and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

### DETAILED DESCRIPTION

Provided herein are pulmonary artery catheters comprising an echocardiogram probe. Devices herein are capable of taking both hemodynamic and echocardiographic measurements.

In some embodiments, catheter devices provided herein comprise an echocardiogram probe (e.g., ultrasound transducer) positioned to reside in the right ventricle of a subject's heart when the distal end of the catheter is within the pulmonary artery. In some embodiments, the distal end of the catheter device comprises an opening attached to a lumen within the catheter to allow for hemodynamic measurements (e.g., within the pulmonary artery). In some embodiments, a catheter device comprises a positioning element (e.g., balloon, sail, etc.) the distal end (e.g., just short of the distal tip) to facilitate proper positioning of the catheter tip in the pulmonary artery (e.g., to wedge the catheter tip in a brand of the pulmonary artery). In some embodiments, a catheter device comprises other elements for making measurements within the vasculature of a subject. In some embodiments, a catheter device comprises various elements for controlling the catheter and/or taking measurements. In some embodiments, provided herein are systems comprising a catheter device herein and other components (e.g., introducer sheath, monitor, computer, syringe, etc.) for operating the catheter and/or taking measurements. In some embodiments, provided herein are methods of inserting/positioning a catheter device herein and/or taking measurements (e.g., intracardiac electrocardiogram, hemodynamic monitoring, etc.) with a catheter device herein.

In some embodiments, catheter devices herein provide continuous, real-time assessment of the heart by providing both hemodynamic (e.g., the dynamics of blood flow and pressures in the heart chambers) and echocardiographic (e.g., information obtained from echocardiogram including visual assessment of heart and ability to assess heart function) in one device. In some embodiments, devices and methods herein provide is a less invasive way of obtaining echocardiographic and hemodynamic information than previous techniques (e.g., having a pulmonary catheter in place and obtaining a snapshot assessment of the heart with echocardiogram (e.g., via transesophageal echocardiogram, intracardiac echocardiogram (via groin insertion), transthoracic echocardiogram, etc.).

In some embodiments, devices herein provide both hemodynamic and echocardiographic assessment of the heart in one device, instead of multiple devices. In some embodiments, devices herein provide real-time, continuous assessment of echocardiography. Using traditional techniques, echocardiography is performed as a ‘snapshot’ of the heart at any given time, as existing devices capable of echocardiographic monitoring are not left in the heart for prolonged time periods.

In some embodiments, provided herein are devices, systems and method capable of making hemodynamic measurements. Classical hemodynamic monitoring is based on the invasive measurement of systemic, pulmonary arterial and venous pressures, and of cardiac output. Since organ blood flow is not directly measured in clinical practice, arterial blood pressure is used as estimate of adequacy of tissue perfusion. There are several different hemodynamic measurements that can be taken to determine different aspects of heart function. Mean arterial pressure (MAP) is the average pressure in a patient's arteries during one cardiac cycle, and is considered a better indicator of perfusion to vital organs than systolic blood pressure (SBP). For example, a MAP of 70 mm Hg may be considered a reasonable target, associated with sign of adequate organ perfusion, in most patients. Central venous pressure (CVP) is the blood pressure in the venae cavae, near the right atrium of the heart, and reflects the amount of blood returning to the heart and the ability of the heart to pump the blood back into the arterial system. Pulmonary wedge pressure (PWP), or cross-sectional pressure (also called the pulmonary arterial wedge pressure (PAWP), pulmonary capillary wedge pressure (PCWP), or pulmonary artery occlusion pressure (PAOP), is the pressure measured by wedging the distal end of a pulmonary artery catheter into a small pulmonary arterial branch. PAOP estimates the left atrial pressure. Normal pressure ranges are described in Table 1.

TABLE 1
Normal hemodynamic pressure ranges for an adult human subject.

| | | Normal |
|---|---|---|
| | | pressure range |
| Site | | (in mmHg) |
| Central venous pressure | | 3-8 |
| Right ventricular pressure | systolic | 15-30 |
| | diastolic | 3-8 |
| Pulmonary artery pressure | systolic | 15-30 |
| | diastolic | 4-12 |
| Pulmonary vein/ | | 2-15 |
| Pulmonary capillary wedge pressure | | |
| Left ventricular pressure | systolic | 100-140 |
| | diastolic | 3-12 |

In certain embodiments, a device described herein is capable of taking any of the aforementioned hemodynamic measurements, in addition to other hemodynamic measurements understood in the field. In some embodiments, devices described herein are particularly suited for PAOP measurements, having a positioning element (e.g., balloon) adjacent to the distal tip of the catheter body that can be wedged into a small pulmonary arterial branch, placing the distal opening in the proper position for such measurements.

In some embodiments, provided herein are devices comprising a catheter body having a distal end for placement within a subject and a proximal end configured to reside outside of the subject, the device comprising (a) a positioning element located adjacent to the distal end of the catheter body and configured for placement of the distal end of the catheter at a desired location within the subject (e.g., into a small pulmonary arterial branch); (b) a distal opening located at the distal end of the catheter body, a distal port extending from the proximal end of the catheter body, and a distal lumen proving fluid communication from the distal opening to the distal port. In some embodiments, the pressure in the environment where the distal opening is placed within a subject (e.g., a small pulmonary arterial branch) can be monitored (e.g., measured) outside of the subject via the connection of the distal opening, distal lumen, and distal port. In some embodiments, the distal port is connected to a hemodynamic monitoring system (e.g., computer, electrocardiograph, etc.) for monitoring/measuring the pressure where the distal opening is placed. In some embodiments, devices herein allow for continuous, real-time monitoring of cardiac pressures (e.g., PAOP).

In some embodiments, provided herein are devices, systems and method capable of intracardiac echocardiogram monitoring. In some embodiments, devise comprise an ultrasound transducer configured for emitting and receive sound waves, converting received sound waves into electrical pulses, and sending electrical pulses through the length of the catheter body via the monitoring wire. In some embodiments, electrical pulses are received by a component of an intracardiac echocardiogram monitoring system and converted into an echocardiogram.

Echocardiograms are typically obtained by one of three methods: transthoracic echocardiogram (TTE), Transesophageal echocardiogram (TEE), and Intracardiac echocardiogram (ICE). Transthoracic is the most common type of echocardiogram and is noninvasive, taking place entirely outside your body. Due to the amount and diversity of tissues between the ultrasound transducer and the heart during TTE, only limited visualization detail is provided by this method. Transesophageal echocardiogram (TEE) is performed by guiding an ultrasound probe into the mouth and down your esophagus. Better images are obtainable, compared to TTE, because the esophagus and heart sit close together within the chest and the sound waves do not need to pass through skin, muscle, or bone. TEE provides greater resolution than TTE, but requires sedation of the subject and therefore is not suitable for taking measurements over a long range of times and carries the additional risks of sedation. Additionally, obesity and lung disease can interfere with standard echocardiography. In classical intracardiac echocardiogram (ICE), a catheter is inserted into a blood vessel near the groin and threaded up to the heart. ICE is often used for placement of devices in the heart and/or during procedures. ICE provides high-resolution real-time visualization of cardiac structures, continuous monitoring of catheter location within the heart, and early recognition of procedural complications, such as pericardial effusion or thrombus formation. None of the existing methods of obtaining electrocardiograms are capable of providing high-resolution, without sedation, and remaining in place over an extended period of time to allow real-time monitoring over a period of days or weeks.

In some embodiments, the devices herein allow insertion of the catheter into a blood vessel (e.g., vein) via an incision in the neck, shoulder, chest, etc. of a subject. For example, in some embodiments, the device is inserted into the jugular vein (e.g., left internal jugular vein, right internal jugular vein, left external jugular vein, right external jugular vein), subclavian vein (e.g., right subclavian vein, left subclavian vein), or femoral vein (e.g. left common femoral vein, right common femoral vein). In some embodiments, the distal tip of the catheter body is guided through the vein, the superior vena cava, right atrium, right ventricle, and into the pulmonary artery of the subject. In some embodiments, when the distal tip of the catheter body is within the pulmonary artery, the ultrasound transducer sits within the right ventricle of the subject.

In some embodiments, provided herein are devices comprising a catheter body having a distal end for placement within a subject and a proximal end configured to reside outside of the subject, the device comprising an ultrasound transducer (electrocardiogram probe) located 10-20 cm from the distal end of the catheter body, and a monitoring wire connected to the ultrasound transducer extending from the proximal end of the catheter body.

In some embodiments, the catheter body, lumens, positioning element, opening, etc., depending on the particular intended application, are composed of modified natural products, metals, ceramics, organic and inorganic materials, modified natural and synthetic polymers, or combinations thereof. In some embodiments, suitable materials are polymeric materials, including derivatives composed of medical-grade fluoroelastomers, polysulfones, polyamides, polyurethanes, polyesters, polyethers, and silicones. For example, most commonly utilized synthetic or modified natural polymeric “biomaterials” include without limitation: polyurethanes, polycarbonates, polyurethane carbonates, polyesters, polyamides, polyimides, polyvinyls, polyolefins, TEFLON, GORETEX, DACRON, polyvinyl alcohols, polyethylene oxides, polyacrylates, polymethacrylates and polycyanoacrylates, latex, polyvinyl chlorides, etc.

In some embodiments, the catheter body is 50-120 cm in length (e.g., 50 cm, 55 cm, 60 cm, 65 cm, 70 cm, 75 cm, 80 cm, 85 cm, 90 cm, 95 cm, 100 cm, 105 cm, 110 cm, 115 cm, 120 cm, or ranges therebetween). In some embodiments, lumens, wires, etc. run from the proximal end of the catheter body, through the interior of the catheter body to their endpoint along the length of the device (e.g., distal tip, 15 cm from the distal tip, etc.). In some embodiments, the catheter body is 3-10 Fr in size (e.g., 3 Fr, 4 Fr, 5 Fr, 6 Fr, 7 Fr, 8 Fr, 9 Fr, 10 Fr, or ranges therebetween. In some embodiments, the catheter body comprises distal end for placement within a subject and a proximal end configured to reside outside of the subject. In some embodiments, the catheter body comprises a polymer material, such as polyvinyl chloride (PVC), thermoplastic polyurethane (TPU), ethylene vinyl acetate copolymers (EVA), nylon ethylene oxide copolymers (PBAX) or blends or copolymer or multi-layer combinations thereof.

In some embodiments, devices herein comprise a positioning element (e.g., distally-located positioning element). In some embodiments, the positioning element is located immediately adjacent to the distal tip (e.g., within 1 cm of the distal tip (e.g., 1 mm, 2 mm, 3 mm, 4 mm, 5 mm, 6 mm, 7 mm, 8 mm, 9 mm, 10 mm, or ranges therebetween). In some embodiments, the positioning element is a balloon or sail. In some embodiments, the positioning element is an inflatable balloon. In some embodiments, the balloon comprises latex or a latex-free material. Ise the balloon is connected to an inflation/deflation lumen that extends from the balloon to the proximal end of the catheter (e.g., extending from the proximal end of the catheter body). In some embodiments, the proximal end of the inflation/deflation lumen comprises a connection element for connecting the lumen (and therefore the balloon) to a controller configured for inflating and deflating the balloon. In some embodiments, the proximal end of the inflation/deflation lumen comprises controller configured for inflating and deflating the balloon. In some embodiments, the balloon controller is a syringe. In some embodiments, the balloon has an inflation volume of between 0.25 and 2.5 ml (e.g., 0.25 ml, 0.5 ml, 1.0 ml, 1.5 ml, 2.0 ml, 2.5 ml, or ranges therebetween).

In some embodiments, devices herein comprise an ultrasound transducer configured for emitting and receive sound waves, to convert received sound waves into electrical pulses, and to send electrical pulses via the monitoring wire. In some embodiments, the ultrasound transducer is located 10-20 cm (e.g., 10 cm, 11 cm, 12 cm, 13 cm, 14 cm, 15 cm, 16 cm, 17 cm, 18 cm, 19 cm, 20 cm, or ranges therebetween (e.g., 12-18 cm, 14-16 cm, etc.) from the distal end of the catheter body, and a monitoring wire connected to the ultrasound transducer extending from the proximal end of the catheter body. In some embodiments, a monitoring wire is configured for connection to a computer system or electrocardiogram monitor. In some embodiments, the monitoring wire places the computer system or electrocardiogram monitor in electric communication with the ultrasound transducer. In some embodiments, the monitoring wire is a coaxial cable.

In some embodiments, devices and systems herein comprise any other suitable elements that are understood in the field of catheters, electrocardiograms, hemodynamic monitoring, or related cardiac monitoring systems/devices.

In some embodiments, a device comprises a thermistor comprising a temperature-sensitive wire within the catheter body that extends from the proximal end of the catheter (e.g., extending beyond the proximal end of the catheter body) to a thermistor bead. In some embodiments, the thermistor bead located 2-6 cm (e.g., 2 cm, 3 cm, 4 cm, 5 cm, 6 cm, or ranges therebetween) from the distal end of the catheter body. In some embodiments, the proximal end of the thermistor is configured for attachment to a cardiac output monitor. In some embodiments, when properly employing in a subject, the terminal portion of the wire, termed the thermistor bead, lies in one of the main pulmonary arteries when the catheter tip is properly positioned. In some embodiments, connection of the thermistor port to a cardiac output monitor allows determination of cardiac output using thermodilution. In some embodiments, a system herein further comprises a cardiac output (CO) monitor.

In some embodiments, a device comprises a proximal opening located 25-35 cm (e.g., 25 cm, 26 cm, 27 cm, 28 cm, 29 cm, 30 cm, 31 cm, 32 cm, 33 cm, 34 cm, 35 cm, or ranges therebetween (e.g., 28-32 cm, etc.) from the distal end of the catheter body, a proximal port extending from the proximal end of the catheter body, a proximal lumen proving fluid communication from the proximal opening to the proximal port. In some embodiments, a proximal injectate port lies within the right atrium when the tip of the catheter is in the pulmonary artery. This port can monitor right atrial pressures pressures (RAP/CVP) and receive the injectate for cardiac output studies. In some embodiments, the proximal port is configured to administer fluids and drugs to the right atrium.

In some embodiments, method are provided herein for hemodynamic and echocardiographic assessment of heart function, for example, after cardiac surgery, when a patient is in heart failure, when a patient is in cardiogenic shock, etc. In some embodiments, the methods herein allow patient care providers to assess in real-time any changes made to medications or devices supporting a patient.

In some embodiments, systems are provided comprising the devise described herein. In some embodiments, systems comprise additional elements understood in the field to be necessary/useful for the employment of the devices herein for their intended purposes.

In some embodiments, systems comprise a catheter insertion kit. In some embodiments, a catheter insertion kit comprises one or more of a needle, guidewire, sheath introducer, and scalpel. In some embodiments, methods are provided for insertion of a catheter device described herein and/or guiding the catheter device to a desired location within a subject. In some embodiments, a device is inserted using the Seldinger technique, modified Seldinger technique, or accelerated Seldinger technique. In some embodiments, a needle is inserted into a vein of the subject through the skin (e.g., using ultrasound guidance if necessary). In some embodiments, a guidewire (e.g., a round-tipped guidewire) is advanced through the lumen of the needle, and the needle is withdrawn. In some embodiments, a scalpel is used to extend the incision around the needle. In some embodiments, a sheath introducer is passed over the guidewire into the blood vessel. In some embodiments, after passing a sheath or tube, the guidewire is withdrawn. In some embodiments, the catheter device is inserted through the introducer sheath.

In some embodiments, systems herein comprise a hemodynamic monitor subsystem. In some embodiments, a hemodynamic monitor subsystem comprises an oscilloscope or computer-implemented oscilloscope, pressure transducer, fluid-filled pressure bag, rigid pressure tubing, stopcock or valve, etc.

In some embodiments, systems herein comprise an electrocardiogram subsystem. In some embodiments, an electrocardiogram subsystem comprises a computer system or electrocardiogram monitor

In some embodiments, systems herein comprise a cardiac output monitor.

### EXPERIMENTAL

The following examples describe real patient encounters that highlight practical applications of embodiments of the devices described herein.

### Example 1

A 51-year-old male patient with severe mitral regurgitation for fifteen years and associated pulmonary hypertension presented in acute decompensated heart failure. He was taken within 24 hours to surgery for mitral valve repair. He had severe right and left ventricular dysfunction weaning from cardiopulmonary bypass which necessitated a temporary left ventricular assist device (device positioned 5 cm into the left ventricle across the aortic valve) and temporary chest closure. Hemodynamic parameters in the ICU include a mean arterial pressure of 55 mmHg on multiple vasopressor and inotropic medications, a pulmonary arterial pressure of 28/12 mmHg, and a central venous pressure of 7 mmHg. Further, the ventricular assist device signaled an alarm for frequent negative pressures in the left ventricle. He was critically unstable and required additional echocardiographic information to help understand his condition.

In this scenario, the mean arterial pressure was low, the patient was decompensating, and an intervention needed to be performed to improve the clinical course. Several scenarios may have accounted for this clinical picture. First, it is difficult to establish whether the pulmonary artery pressure represents a weak right ventricle that is unable to generate a higher pulmonary artery pressure than 28/12 mmHg, or a well decompressed right ventricle but instead an issue of appropriate ventricular assist device positioning. A number of potential interventions, some contradictory to each other, may have been necessary to improve the clinical condition.

The transthoracic echocardiogram had poor visualization of the heart due to the temporary chest closure and only provided a brief snapshot of the patient, which is not useful when ongoing monitoring is required. A transesophageal echocardiogram was too invasive, requiring safe passage of the probe down the esophagus of an intubated patient. Both echocardiograms would require another provider to perform the duty, a capability most hospitals do not have at all times of the day. Ultimately, interventions to improve the patient's clinical condition were based on pressure hemodynamic information alone.

In this scenario the devices described herein offer the following advantages:
1) Provide visual assessment of right and left ventricle function (can assess visual wall contractility, degree of ventricle dilation, and ejection fraction);
2) Assess the mitral valve repair for any surgical issues which may need operating room correction;
3) Assess proper position of the ventricular assist device (e.g., if it is “too deep” in the left ventricle or “too shallow” which can be adjusted easily at the bedside);
4) Intracardiac echocardiography offers the best visualization of the heart compared to transthoracic and transesophageal echocardiography; and
5) Provide continuous monitoring, which would allow for easily assessing the response to clinical interventions performed.

### Example 2

A 45-year-old man with multivessel coronary artery disease and mild ischemic mitral regurgitation presented to the hospital with non-ST elevation myocardial infarction. He underwent urgent coronary artery bypass grafting. The evening of the date of surgery in the ICU the patient required multiple vasopressor and inotropic medication to support his blood pressure and cardiac output. He had elevated right and left diastolic filling pressures and a low cardiac output based on pulmonary artery pressure measurements. He was critically unstable and required an intervention but the differential diagnosis in this scenario included post-cardiotomy cardiogenic shock, worsening ischemic mitral regurgitation, ongoing myocardial infarction, cardiac tamponade, or several causes for distributive or obstructive shock that could not be further elucidated without echocardiographic information.

A bedside transthoracic echocardiogram was obtained to provide further information. A patient technician had to be called in from home to perform the bedside echocardiogram which took 2.5 hours to complete from the time it was requested. By this time the patient had more acutely decompensated. The bedside echocardiogram identified a large effusion surrounding the heart, but it had limited visualization of cardiac structures due to large body habitus and had limited diagnostic capability. Based on these findings the presumptive diagnosis was cardiac tamponade and the patient had to be brought emergently to the operating room. The patient was reintubated in order to perform a transesophageal echocardiogram which demonstrated evidence of right ventricular collapse and large pericardial effusion consistent with cardiac tamponade, fluid and clots around the heart which exerts external pressure to heart and reduces it's pumping capability. The patient underwent a chest re-exploration to remove blood clots surrounding the heart and the patient improved hemodynamically.

In this scenario, a device described herein (e.g., a catheter with echocardiography probe) would offer several advantages:
1) Allow more prompt diagnosis of tamponade and identify heart chamber echocardiographic signs of tamponade with better visualization than alternate echocardiography modalities;
2) Assess the response to medical therapies to support tamponade prior to definitive surgical evacuation of blood surrounding the heart (e.g., administering IV fluids to temporize tamponade); and
3) Assess any residual re-accumulation of blood surrounding the heart after the surgical procedure (which can occur in the setting of coagulopathy or unidentified source of bleeding).

1. A device comprising a catheter body having a distal end for placement within a subject and a proximal end configured to reside outside of the subject, the device comprising:
(a) a positioning element located adjacent to the distal end of the catheter body and configured for placement of the distal end of the catheter at a desired location within the subject;
(b) a distal opening located at the distal end of the catheter body, a distal port extending from the proximal end of the catheter body, and a distal lumen proving fluid communication from the distal opening to the distal port; and
(c) an ultrasound transducer located 10-20 cm from the distal end of the catheter body, and a monitoring wire connected to the ultrasound transducer extending from the proximal end of the catheter body.

2. The device of claim 1, wherein the distally-located positioning element is an inflatable balloon.

3. The device of claim 2, wherein the balloon is connected to an inflation/deflation lumen that extends from the balloon to the proximal end of the catheter.

4. The device of claim 3, comprising a connection at the proximal end of the inflation/deflation lumen for attachment to a balloon controller configured for inflating and deflating the balloon.

5. The device of claim 3, comprising a balloon controller at the proximal end of the inflation/deflation lumen for inflating and deflating the balloon.

6. The device of claim 4, wherein the balloon controller is a syringe.

7. The device of claim 6, wherein the balloon has an inflation volume of between 0.25 and 2.5 ml.

8. The device of claim 1, wherein the diameter of the distal opening is 0.50-2.0 mm in diameter.

9. The device of claim 1, wherein the distal lumen is 0.50-2.0 mm in diameter.

10. The device of claim 1, wherein the distal port is configured for connection to a hemodynamic monitoring system.

11. The device of claim 1, wherein the ultrasound transducer located 14-16 cm from the distal end of the catheter body.

12. The device of claim 11, wherein the ultrasound transducer located 15 cm cm from the distal end of the catheter body.

13. The device of claim 1, wherein ultrasound transducer is configured for emitting and receive sound waves, and to convert received sound waves into electrical pulses.

14. The device of claim 1, wherein the monitoring wire is configured for connection to a computer system or electrocardiogram monitor.

15. The device of claim 14, wherein the monitoring wire places the computer system or electrocardiogram monitor in electric communication with the ultrasound transducer.

16. The device of claim 1, wherein the monitoring wire is a coaxial cable.

17. The device of claim 1, further comprising a thermistor comprising a temperature-sensitive wire within the catheter body that extends from the proximal end of the catheter to a thermistor bead located 2-6 cm from the distal end of the catheter body.

18. (canceled)

19. The device of claim 1, further comprising a proximal opening located 25-35 cm from the distal end of the catheter body, a proximal port extending from the proximal end of the catheter body, and a proximal lumen proving fluid communication from the proximal opening to the proximal port.

20. A method of cardiac monitoring comprising:
(a) placing the device of claim 1 within the heart of a subject such that the distal tip of the catheter body is within the pulmonary artery of the subject and the ultrasound transducer is within the right ventricle of the subject;
(b) monitoring hemodynamic pressure using the distal opening, distal lumen, and distal port; and
(c) obtaining an echocardiogram using the ultrasound transducer and monitoring wire.

21.-31. (canceled)

32. A system comprising the device of claim 1 and one or more of:
(i) a catheter insertion kit;
(ii) a hemodynamic monitor subsystem;
(iii) an electrocardiogram subsystem; and
(iv) a cardiac output monitor.

33.-35. (canceled)
